(19) United States
(12) Patent Application Publication
(10) Pub. No.: US 2016/0206210 A1
(43) Pub. Date: Jul. 21, 2016

(54) OPTICAL MEASUREMENT APPARATUS

(71) Applicant: OLYMPUS CORPORATION, Tokyo (JP)

(72) Inventors: Seiki TORIYAMA, Hino-shi (JP); Yoshimine KOBAYASHI, Hachioji-shi (JP); Seigo ITO, Hachioji-shi (JP)

(73) Assignee: OLYMPUS CORPORATION, Tokyo (JP)

(21) Appl. No.: 15/084,254

(22) Filed: Mar. 29, 2016

Related U.S. Application Data
(63) Continuation of application No. PCT/JP2015/060237, filed on Mar. 31, 2015.
(60) Provisional application No. 61/982,099, filed on Apr. 21, 2014.

(51) Int. Cl. A61B 5/00

(52) U.S. Cl. CPC A61B 5/0084; A61B 2090/306

(57) ABSTRACT

Provided is an optical measurement apparatus to which a measurement probe having an illumination fiber and a light receiving fiber is configured to be detachably connected and which is configured to perform optical measurement on a measurement target. The optical measurement apparatus includes: a light source unit configured to emit illumination light to the measurement target via the illumination fiber; a light receiving unit configured to receive light propagated via the light receiving fiber; and a calculation unit configured to calculate a characteristic value of a measurement history of each of the illumination light and external light incident from outside, based on a plurality of measured values obtained by receiving diffused light of the illumination light reflected from a reflection index member and on a plurality of measured values obtained by receiving diffused light of the external light that has been transmitted through a diffusion index member as uniform light.

### CROSS-REFERENCE TO RELATED APPLICATIONS

This application is a continuation of PCT international application Ser. No. PCT/JP2015/060237 filed on Mar. 31, 2015 which designates the United States, incorporated herein by reference, and which claims the benefit of priority from U.S. provisional application No. 61/982,099 filed on Apr. 21, 2014, incorporated herein by reference.

### BACKGROUND

1. Technical Field

The disclosure relates to an optical measurement apparatus for measuring optical characteristics of a body tissue.

2. Related Art

Conventionally, an optical measurement apparatus has been known to irradiate a measurement target with light from a measurement probe and measures, based on a result of measuring the light reflected from the measurement target, optical characteristics of the measurement target. In order to ensure measurement accuracy of the result of measurement, the optical measurement apparatus needs to perform a calibration process before starting the measurement of the measurement target. As a technique of performing such a calibration process, a technique is known to perform measurement with a distal end portion of a measurement probe being inserted into an adjustment tool accommodating a calibration member having a known measured value and to perform, based on a result of this measurement, a calibration process of an optical measurement apparatus (see Japanese Patent Application Laid-open No. 2012-139446).

### SUMMARY

In some embodiments, provided is an optical measurement apparatus to which a measurement probe having an illumination fiber and a light receiving fiber is configured to be detachably connected and which is configured to perform optical measurement on a measurement target. The optical measurement apparatus includes: a light source unit configured to emit illumination light to the measurement target via the illumination fiber; a light receiving unit configured to receive light propagated via the light receiving fiber; and a calculation unit configured to calculate a characteristic value of a measurement history of each of the illumination light and external light incident from outside, based on a plurality of measured values obtained by receiving, by use of the light receiving unit via the light receiving fiber, diffused light of the illumination light reflected from a reflection index member and on a plurality of measured values obtained by receiving, by use of the light receiving unit via the light receiving fiber, diffused light of the external light that has been transmitted through a diffusion index member as uniform light.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a perspective diagram illustrating a schematic configuration of an optical measurement system according to a first embodiment of the present invention;

FIG. 2 is a block diagram illustrating a functional configuration of the optical measurement system according to the first embodiment of the present invention;

FIG. 3 is a perspective diagram illustrating a schematic configuration of a calibration module used in the optical measurement system according to the first embodiment of the present invention;

FIG. 4 is a cross sectional view taken along the lines iv-iv of FIG. 3;

FIG. 5 is a schematic diagram illustrating an outline of a determination method executed by the optical measurement system according to the first embodiment of the present invention;

FIG. 6 is a schematic diagram illustrating an outline of a determination method for the determination unit of the optical measurement system according to the first embodiment of the present invention to determine whether or not a characteristic value of a measurement history of illumination light calculated by a calculation unit has a falling trend;

FIG. 7 is a schematic diagram illustrating an outline of a calculation method for a threshold value calculated by a threshold value calculation unit of the optical measurement system according to the first embodiment of the present invention;

FIG. 8 is a diagram illustrating an example of an abnormality information table related to abnormality and degradation information recorded by the abnormality information recording unit according to the first embodiment of the present invention;

FIG. 9 is a flow chart illustrating an outline of a degradation and abnormality determination process executed by the optical measurement system according to the first embodiment of the present invention;

FIG. 10 is a block diagram illustrating a functional configuration of an optical measurement system according to a second embodiment of the present invention;

FIG. 11 is a diagram illustrating an example of an abnormality information table related to abnormality information recorded by the abnormality information recording unit according to the second embodiment of the present invention; and

FIG. 12 is a diagram illustrating another example of the abnormality information table related to the abnormality information recorded by the abnormality information recording unit according to the second embodiment of the present invention.

### DETAILED DESCRIPTION

Modes for carrying out the present invention (hereinafter, referred to as “embodiments”) will be described below with reference to the drawings. The same reference signs are used to designate the same elements throughout the drawings. It is noted that the drawings are schematic, and a relation between a thickness and a width of each component, ratios among the respective components, and the like are different from the actual. Moreover, a portion is included, which has different dimensional relations and ratios among the drawings. The present invention is not limited by the embodiments.

### First Embodiment

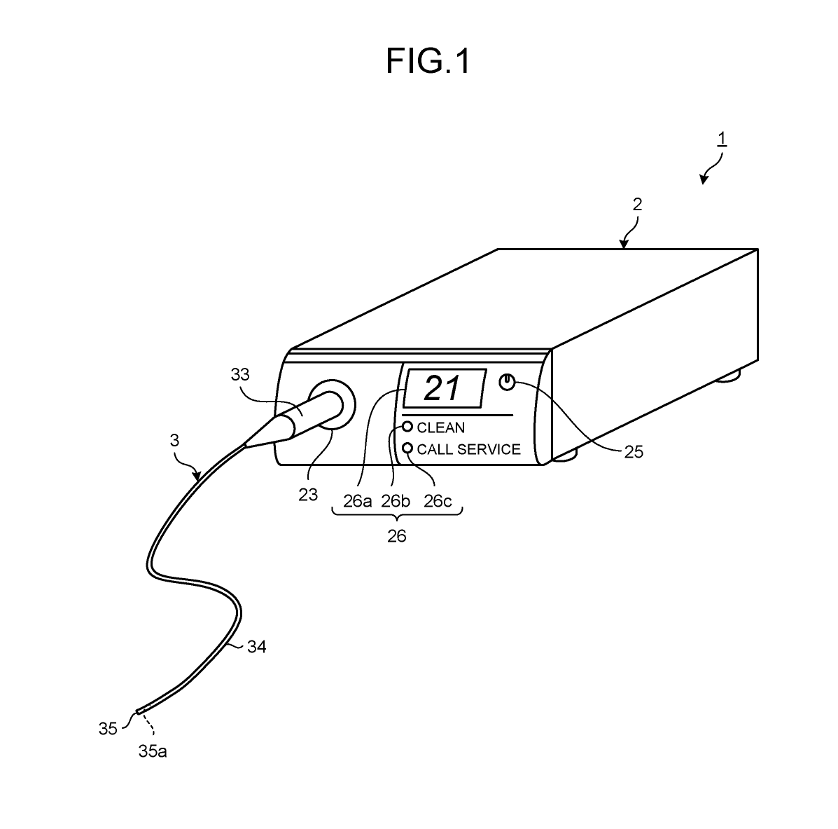

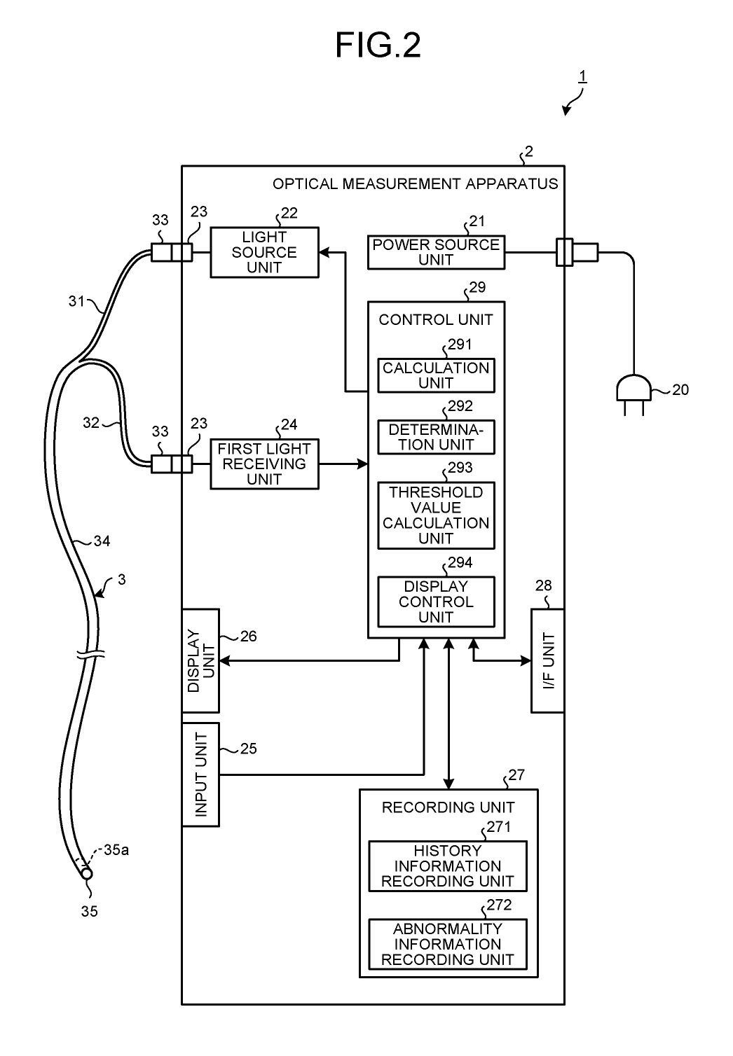

FIG. 1 is a perspective diagram illustrating a schematic configuration of an optical measurement system according to a first embodiment of the present invention. FIG. 2 is a block diagram illustrating a functional configuration of the optical measurement system according to the first embodiment of the present invention.

An optical measurement system 1 illustrated in FIG. 1 and FIG. 2 includes: an optical measurement apparatus 2 that performs optical measurement on a measurement target, such as a body tissue, which is a scatterer, and detects properties (characteristics) of the measurement target; and a measurement probe 3 that is freely attachable and detachable to and from the optical measurement apparatus 2 and is introduced into a subject.

First, a configuration of the optical measurement apparatus 2 will be described.

The optical measurement apparatus 2 includes a commercial power source connector 20, a power source unit 21, a light source unit 22, a connection unit 23, a first light receiving unit 24, an input unit 25, a display unit 26, a recording unit 27, an I/F unit 28, and a control unit 29.

The power source unit 21 converts an electric power input via the commercial power source connector 20 into a predetermined voltage, and supplies this converted electric power to each unit of the optical measurement apparatus 2.

The light source unit 22 emits illumination light to the measurement probe 3 via the connection unit 23. The light source unit 22 is realized by using an incoherent light source, such as a white light emitting diode (LED), a tungsten lamp, and a halogen lamp, and as necessary, one lens or a plurality of lenses. As such a lens, for example, a condenser lens or a collimator lens can be mentioned.

The connection unit 23 detachably connects the measurement probe 3 to the optical measurement apparatus 2. The connection unit 23 outputs the illumination light emitted from the light source unit 22 to the measurement probe 3 and outputs returned light of the illumination light incident via the measurement probe 3 to the first light receiving unit 24. The connection unit 23 is realized by using, for example, a sub-miniature type A (SMA) connector and an optical fiber.

The first light receiving unit 24 receives and measures light propagated via the connection unit 23 and the measurement probe 3. Specifically, the first light receiving unit 24 generates and outputs to the control unit 29 a measurement result of the measurement target by receiving and performing photoelectric conversion on the returned light of the illumination light incident from the measurement probe 3 via the connection unit 23. The first light receiving unit 24 is realized by using an imaging element, such as a charge coupled device (CCD) or a complementary metal oxide semiconductor (CMOS). In the first light receiving unit 24, a spectrometer, which receives the returned light of the illumination light incident from the measurement probe 3 and is able to disperse spectral components of the received returned light of the illumination light, may be provided.

The input unit 25 receives various information of the optical measurement apparatus 2, such as a start signal for instructing start of the measurement. The input unit 25 is configured by using a touch panel, push-type buttons, or the like.

The display unit 26 displays, under control by the control unit 29, various information of the optical measurement apparatus 2. Specifically, the display unit 26 has: a display monitor 26a that displays abnormality information related to the optical measurement system 1; a clean lamp 26b that instructs a user to do cleaning; and a call lamp 26c that instructs the user to contact a serviceman. Further, the display unit 26 outputs the measurement result of the measurement target and operational information related to the optical measurement apparatus 2. The display unit 26 is realized by using a liquid crystal or organic electroluminescence display panel or the like, a display lamp that is able to blink, a speaker or the like that outputs a sound, and the like. In the first embodiment, the display unit 26 functions as an output unit.

The recording unit 27 records various programs for operating the optical measurement apparatus 2 and various data or the like used for the optical measurement apparatus 2. Further, the recording unit 27 has: a history information recording unit 271 that records a past measurement result in a calibration process; and an abnormality information recording unit 272 that records abnormality information in which predicted abnormalities, characteristic values of measurement histories of each of the illumination light and external light estimated when these abnormalities occur, and contents displayed on the display unit 26 are associated with one another. The predicted abnormalities are abnormalities due to optical performance and abnormalities due to structural defects, of each of the measurement probe 3 and the light source unit 22. This abnormality due to optical performance is an abnormality caused by degradation due to a decrease in transmittance of the measurement probe 3 or degradation due to a decrease in light quantity of the light source unit 22. Further, these abnormalities due to structural defects are abnormalities caused by breakage of the measurement probe 3, a stain adhered to a distal end portion of the measurement probe 3, failure of the first light receiving unit 24, uncleanness of a later described calibration module, and the like. The recording unit 27 is realized by using a volatile memory, a non-volatile memory, and the like. Details of the abnormality information recorded by the abnormality information recording unit 272 will be described later.

The I/F unit 28 outputs a signal input from outside to the control unit 29. Specifically, the I/F unit 28 outputs to the control unit 29, a signal input from an endoscope apparatus, for example, a signal indicating an irradiation timing of illumination light emitted by the endoscope apparatus, an instruction signal input from an operating unit of the endoscope apparatus, or the like. Further, the I/F unit 28 outputs the measurement result measured by the optical measurement apparatus 2 to the endoscope apparatus.

The control unit 29 comprehensively controls operations of processes of respective units of the optical measurement apparatus 2. The control unit 29 is configured by using a central processing unit (CPU) or the like, and controls the optical measurement apparatus 2 by performing transfer or the like of instruction information and data with respect to each unit of the optical measurement apparatus 2. Further, the control unit 29 has a calculation unit 291, a determination unit 292, a threshold value calculation unit 293, and a display control unit 294.

The calculation unit 291 calculates a characteristic related to a property of the measurement target, based on measurement data of the returned light of the illumination light input from the first light receiving unit 24. Further, the calculation unit 291 calculates the characteristic value of the measurement history of each of the illumination light and the external light, based on a plurality of measured values recorded by the history information recording unit 271. The plurality of measured values is measured values of each of: diffused light based on the illumination light reflected from a reflection index member irradiated with the illumination light received by the first light receiving unit 24 via a light receiving fiber; and diffused light based on light incident from outside received by the first light receiving unit 24 via a diffusion index member that transmits light incident from outside as uniform light and via the light receiving fiber.

The determination unit 292 determines, based on a calculation result of the calculation unit 291 and the abnormality information recorded by the abnormality information recording unit 272, whether or not an abnormality has occurred in any of the measurement probe 3, the light source unit 22, and the first light receiving unit 24.

The threshold value calculation unit 293 calculates a threshold value used when the determination unit 292 determines the characteristic (trend) of the measurement history of each of: the measured values of the diffused light based on the illumination light; and the measured values of the diffused light based on the external light incident from the outside, these measured values being recorded by the history information recording unit 271.

The display control unit 294 controls a display mode of the display unit 26. Specifically, based on a determination result of the determination unit 292, information related to an abnormality in the optical measurement system 1 is caused to be displayed on the display monitor 26a. Further, the display control unit 294 lights the clean lamp 26b or the call lamp 26c, based on the determination result of the determination unit 292.

Next, a configuration of the measurement probe 3 will be described.

The measurement probe 3 is configured by using at least two optical fibers. Specifically, the measurement probe 3 is configured by using an illumination fiber 31 (illumination channel) that irradiates the measurement target with illumination light, and a first light receiving fiber 32 (first light receiving Ch) that receives returned light of the illumination light reflected and/or scattered from the measurement target. The measurement probe 3 includes: a proximal end portion 33 that is detachably connected to the connection unit 23; a flexible portion 34 that has flexibility; and a distal end portion 35 that emits the illumination light supplied via the connection unit 23 from the light source unit 22 and receives the returned light of the illumination light from the measurement target. Further, at the distal end portion 35, a rod lens 35a is provided for keeping a distance between the measurement target and the distal end portion 35 constant.

The optical measurement system 1 configured as described above performs measurement of the measurement target, after performing the calibration process. In this calibration process, the later described calibration module is used.

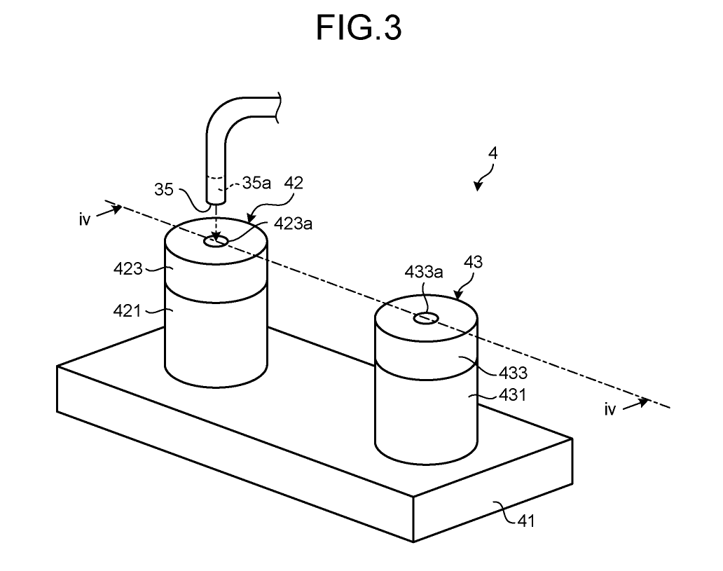

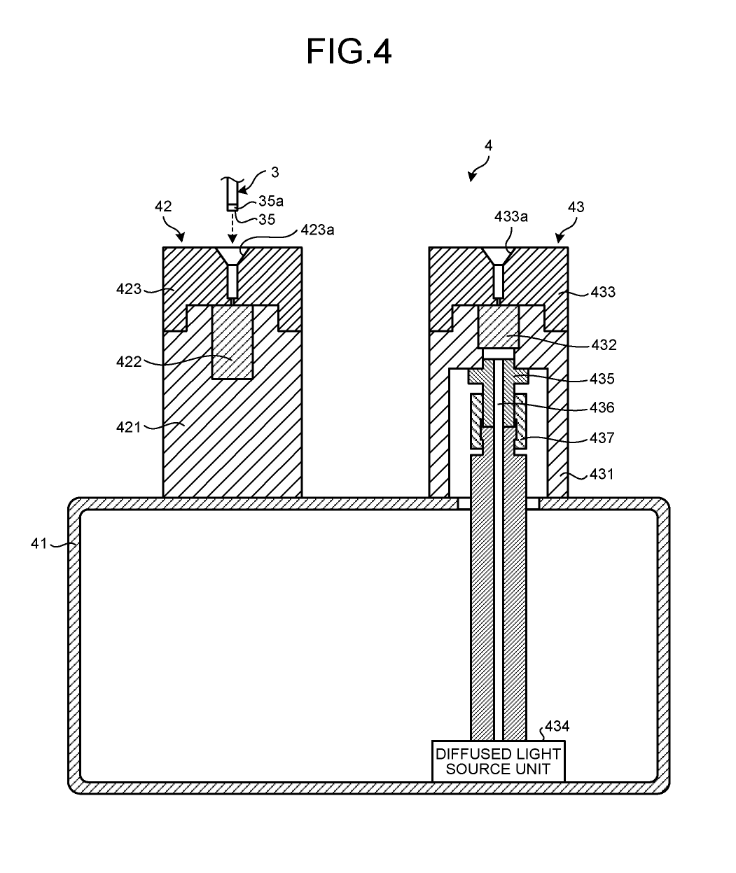

FIG. 3 is a perspective diagram illustrating a schematic configuration of the calibration module used in the optical measurement system 1. FIG. 4 is a cross sectional view taken along the lines iv-iv of FIG. 3.

A calibration module 4 illustrated in FIG. 3 and FIG. 4 has a base portion 41 having a cavity inside thereof, a reflection index calibration unit 42, and an emission index calibration unit 43. The reflection index calibration unit 42 and the emission index calibration unit 43 are provided fixed to the base portion 41.

First, a configuration of the reflection index calibration unit 42 will be described.

The reflection index calibration unit 42 has a reflection index main body portion 421, a reflection index member 422, and a reflection lid portion 423.

The reflection index main body portion 421 is columnar and inside thereof, the reflection index member 422 is accommodated. On a top surface of the reflection index main body portion 421, the reflection lid portion 423 is detachably attached.

The reflection index member 422 is a member that has a white plate or a surface having a high reflectivity with respect to the illumination light. When the calibration process of the optical measurement system 1 is performed, the reflection index member 422 is an irradiation object to be irradiated with the illumination light from a distal end of the measurement probe 3.

The reflection lid portion 423 is approximately tubular and has an insertion portion 423a through which the measurement probe 3 is insertable. The reflection lid portion 423 may be formed by using an elastic body, for example, rubber, silicone rubber, or the like.

Next, a configuration of the emission index calibration unit 43 will be described.

The emission index calibration unit 43 has an emission index main body portion 431, a diffusion index member 432, an emission lid portion 433, a diffused light source unit 434, a connector portion 435, an optical fiber 436, and a sleeve 437.

The emission index main body portion 431 is approximately tubular. Inside the emission index main body portion 431, a diffusion index member 432 is accommodated.

The diffusion index member 432 moderates non-uniformity of luminance of light emitted from the diffused light source unit 434 via the optical fiber 436 and transmits uniform light (even light).

The emission lid portion 433 is approximately tubular and has an insertion portion 433a through which the measurement probe 3 is insertable. The emission lid portion 433 may be formed by using an elastic body, for example, rubber, silicone rubber, or the like.

The diffused light source unit 434 emits illumination light to the diffusion index member 432 via the optical fiber 436. The diffused light source unit 434 is configured by using a white LED or the like.

The connector portion 435 holds the optical fiber 436 inside thereof. One face of the connector portion 435 is in contact with and screwed to the emission index main body portion 431, and another face thereof holds, by the sleeve 437, the optical fiber 436 that guides the light from the diffused light source unit 434.

In the calibration module 4 configured as described above, the calibration process is executed with the measurement probe 3 being inserted into the insertion portion 423a of the reflection index calibration unit 42 or the insertion portion 433a of the emission index calibration unit 43.

Specifically, if the measurement probe 3 is inserted in the insertion portion 423a of the reflection index calibration unit 42, the control unit 29 causes the light source unit 22 to emit the illumination light to irradiate the reflection index member 422 with the illumination light via the measurement probe 3. Thereafter, the calculation unit 291 calculates a measured value (hereinafter, simply referred to as “measured value of illumination light”) of the diffused light based on the illumination light reflected by the reflection index member 422 received by the first light receiving unit 24 via the measurement probe 3, and based on a result of this calculation, executes a calibration process of the optical measurement system 1.

Subsequently, if the measurement probe 3 is inserted in the insertion portion 433a of the emission index calibration unit 43, the calculation unit 291 calculates a measured value of the diffused light (hereinafter, simply referred to as “measured values of external light”) incident from the diffused light source unit 434 received by the first light receiving unit 24 via the diffusion index member 432 and the measurement probe 3, and based on a result of this calculation, executes a calibration process of the optical measurement system 1. Each of the measured value of illumination light and the measured value of external light is sequentially recorded in the history information recording unit 271 every time a calibration process is executed.

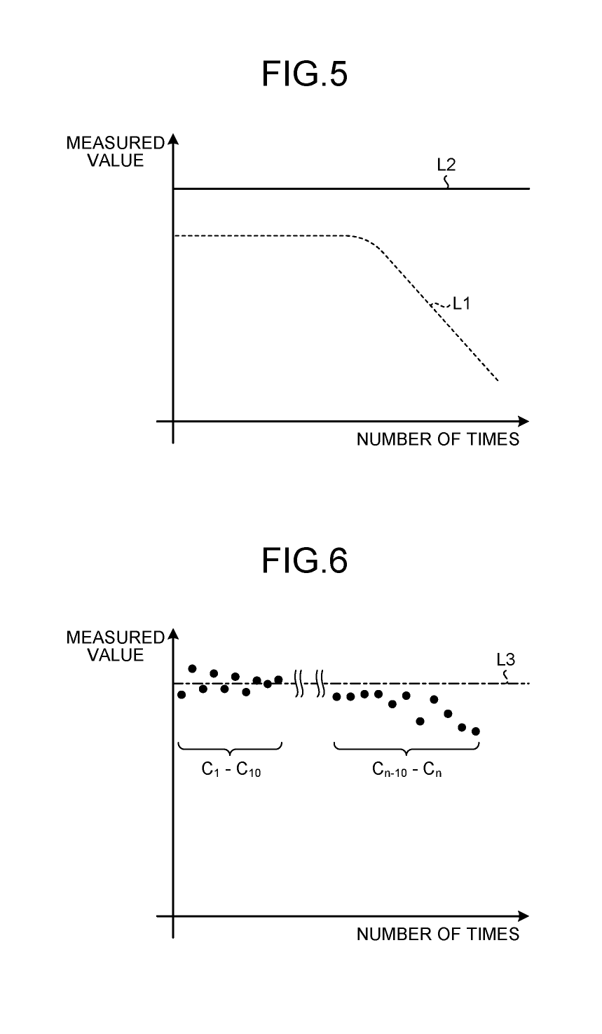

Next, an abnormality determination process executed by the optical measurement system 1 will be described. FIG. 5 is a schematic diagram illustrating an outline of a determination method executed by the determination unit 292. In FIG. 5, a horizontal axis represents the number of times, and a vertical axis represents measured value of light received by the first light receiving unit 24. Further, in FIG. 5, a line L1 represents the characteristic value of the measurement history of the illumination light calculated by the calculation unit 291 and a straight line L2 represents the characteristic value of the measurement history of the external light (illumination light) calculated by the calculation unit 291.

As illustrated in FIG. 5, the determination unit 292 determines that the measurement probe 3 is normal and determines that the light quantity of the light source unit 22 has decreased (the light source unit 22 has degraded), that is, that the light source unit 22 is abnormal, if a falling trend is seen as the characteristic value of the measurement history of the measured values of the illumination light calculated by the calculation unit 291 (see the line L1) and if an increasing or decreasing trend is not seen as the characteristic value of the measurement history of the measured values of the external light (see the straight light L2). The characteristic value of the measurement history is a slope of a straight line approximated based on a plurality of the measured values for the illumination light or a plurality of the measured values for the external light.

A determination method, by which the determination unit 292 determines whether or not the characteristic value of the measurement history calculated by the calculation unit 291 is of a falling trend, will be described. FIG. 6 is a schematic diagram illustrating an outline of the determination method when the determination unit 292 determines whether or not the characteristic value of the measurement history of the illumination light calculated by the calculation unit 291 is of the falling trend. In FIG. 6, a horizontal axis represents the number of times, and a vertical axis represents the measured value of light received by the first light receiving unit 24. Further, in FIG. 6, a straight line L3 represents a threshold value used when the falling trend of the measured values of the illumination light is determined by the determination unit 292. In FIG. 6, the method of determining the falling trend for the characteristic value of the measurement history of the illumination light will be described, and since a similar process may be used when a falling trend or a rising trend for the characteristic value of the measurement history of the external light is determined, description thereof will be omitted.

As illustrated in FIG. 6, in order to eliminate contingent elements, the threshold value calculation unit 293 calculates an average value Cₐᵥ used in determining the falling trend of the measured values of the illumination light, by using an average value of the first ten measurement results (C₁ to C₁₀) recorded in the history information recording unit 271.

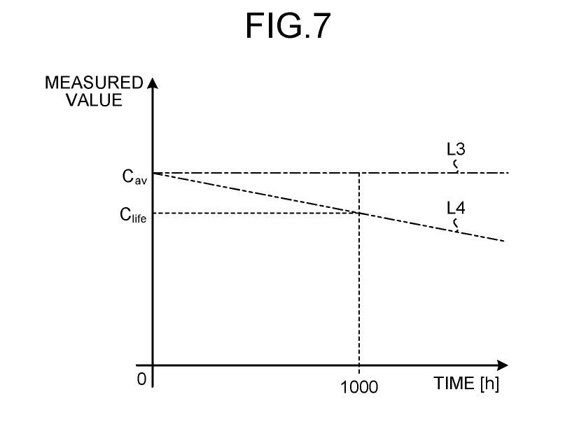

Further, the threshold value calculation unit 293 calculates the threshold value considering aging degradation of the light source unit 22. FIG. 7 is a schematic diagram illustrating an outline of a method of calculating the threshold value calculated by the threshold value calculation unit 293. In FIG. 7, a horizontal axis represents time, and a vertical axis represents the measured value of light received by the first light receiving unit 24. Further, in FIG. 7, the straight line L3 represents the average value Cₐᵥ, and a straight line L4 represents a threshold value C_{life} obtained by taking into account the aging degradation of the light source unit 22 with respect to the average value.

As illustrated in FIG. 7, the threshold value calculation unit 293 calculates the threshold value C_{life} by taking into account the aging degradation of the light source unit 22 with respect to the average value Cₐᵥ. Specifically, the threshold value calculation unit 293 calculates the threshold value C_{life} by the following equation, if attenuation (degradation) of the light source unit 22 is 5% per 1000 hours and the time elapsed is “t”.

C_{life}=(0.95/1000)×t×Cₐᵥ (1)

As described above, the threshold value calculation unit 293 eliminates the contingent elements and calculates the threshold value C_{life} in consideration of the aging degradation of the light source unit 22. The determination unit 292 may use the average value Cₐᵥ calculated by the threshold value calculation unit 293 as the threshold value.

The determination unit 292, as illustrated in FIG. 6, determines whether or not, going back in order from the measured value Cₙ (n=natural number) to the measured value Cₙ₋₁₀, C_{life}−Cₙ>0 and if C_{life}−Cₙ>0, determines that the measured values of the illumination light has the falling trend. That is, the determination unit 292 determines that an abnormality has occurred in the light quantity of the illumination light of the light source unit 22, even if the measured values of the illumination light has increased up or decreased down over some range, if the characteristic value of the measurement history of the measured values of the illumination light calculated by the calculation unit 291 is of the falling trend and the threshold value C_{life} has not been exceeded even once over the ten times. The above described number of times and a ratio of the attenuation for calculating the threshold value and the number of times used for the determination may be changed as appropriate.

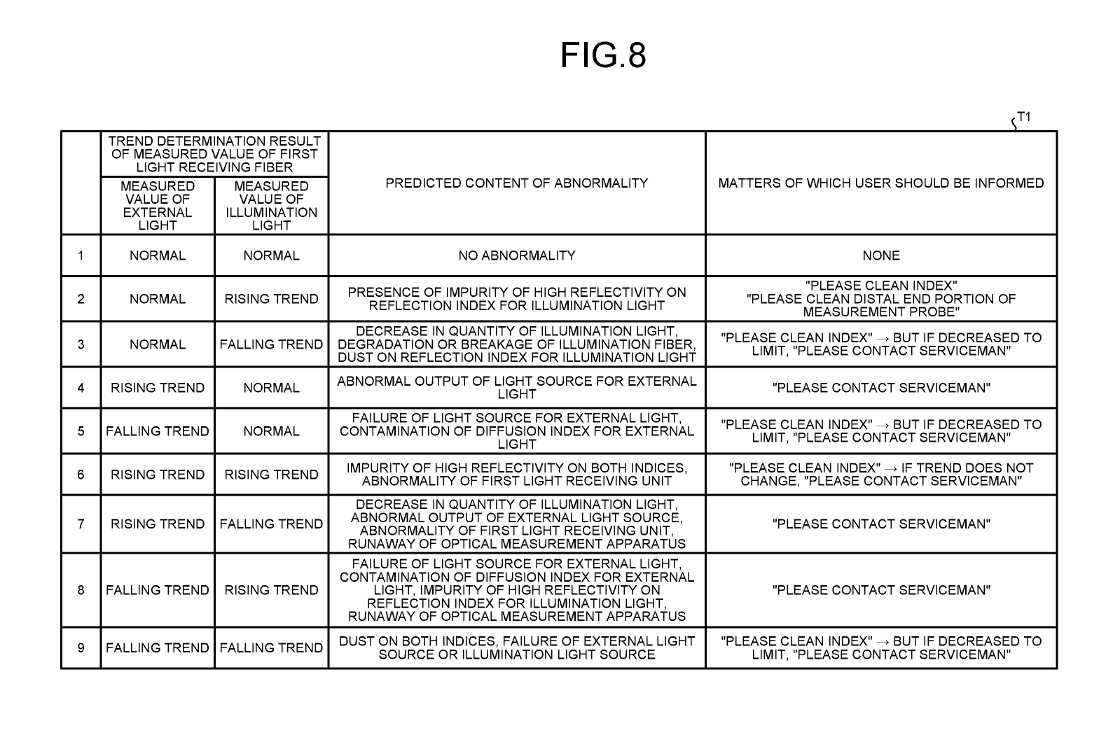

Next, degradation and abnormality patterns determined by the determination unit 292 will be described. FIG. 8 is a diagram illustrating an example of an abnormality information table related to the abnormality information recorded by the abnormality information recording unit 272.

In an abnormality information table T1 illustrated in FIG. 8, the characteristic values of the measurement histories by the calculation unit 291 (the measured values of the external light and the measured values of the illumination light), predicted contents of abnormality, and matters of which a user should be informed, are recorded in association with one another. Specifically, for “1” in the abnormality information table T1, if the characteristic value (trend) of the measurement history for the measured values of the external light is “Normal” and the characteristic value of the measurement history for the measured values of the illumination light is “Normal”, the predicted content of abnormality is recorded as “No abnormality” and the matters of which a user should be informed is recorded as “None”. That is, the determination unit 292 determines that the optical measurement system 1 is normal, if each of the characteristic value of the measurement history for the measured values of the external light and the characteristic value of the measurement history for the measured values of the illumination light, which are calculated by the calculation unit 291, is normal.

For “2” of the abnormality information table T1, if the characteristic value of the measurement history for the measured values of the external light is “Normal” and the characteristic value of the measurement history for the measured values of the illumination light is of “Rising trend”, the predicted content of abnormality is recorded as “Presence of impurity of high reflectivity on reflection index for illumination light” and the matters of which a user should be informed is recorded as “Please clean index”. That is, the determination unit 292 determines that an abnormality has occurred in the reflection index member 422 if the characteristic value of the measurement history for the measured values of the external light calculated by the calculation unit 291 is normal and the characteristic value of the measurement history for the measured values of reflected light of the illumination light is of the rising trend. Further, the display control unit 294 causes the clean lamp 26b of the display unit 26 to be lighted and causes the display monitor 26a to display information indicating the content of abnormality. For “2” of the abnormality information table T1, since the characteristic value of the illumination light similarly indicates the rising trend when an impurity of a high reflectivity adheres to the distal end portion 35 of the measurement probe 3, for the matters of which a user should be informed, “Please clean distal end portion 35 of measurement probe 3” is also recorded.

For “3” of the abnormality information table T1, if the characteristic value of the measurement history for the measured values of the external light is “normal” and the characteristic value of the measurement history for the measured values of the illumination light is of a “falling trend”, the predicted content of abnormality is recorded as “Decrease in quantity of illumination light, degradation or breakage of illumination fiber, dust on reflection index for illumination light” and the matters of which a user should be informed is recorded as “Please clean index”. That is, the determination unit 292 determines that any one or more of: an abnormality in the light source unit 22; an abnormality in the illumination fiber 31; and an abnormality in the reflection index member 422 has occurred, if the characteristic value of the measurement history for the measured values of the external light calculated by the calculation unit 291 is normal and the characteristic value of the measurement history for the measured values of the illumination light is of the falling trend. In this case, the display control unit 294 causes the clean lamp 26b of the display unit 26 to be lighted and causes the display monitor 26a to display information indicating the content of abnormality. For “3” of the abnormality information table T1, if no improvement is achieved even if cleaning of the index is performed, because maintenance of the light source unit 22 becomes necessary, as the matters of which a user should be informed, “Please contact serviceman” is recorded.

For “4” of the abnormality information table T1, if the characteristic value of the measurement history for the measured values of the external light is of a “rising trend” and the characteristic value of the measurement history for the measured values of the illumination light is “normal”, the predicted content of abnormality is recorded as “Abnormal output of light source for external light” and the matters of which a user should be informed is recorded as “Please contact serviceman”. That is, the determination unit 292 determines that an abnormality has occurred in the diffused light source unit 434 if the characteristic value of the measurement history for the measured values of the external light calculated by the calculation unit 291 is of the rising trend and the characteristic value of the measurement history for the measured values of the illumination light is normal. In this case, the display control unit 294 causes the call lamp 26c of the display unit 26 to be lighted and causes the display monitor 26a to display information indicating the content of abnormality.

For “5” of the abnormality information table T1, if the characteristic value of the measurement history for the measured values of the external light is of a “falling trend” and the characteristic value of the measurement history for the measured values of the illumination light is “normal”, the predicted content of abnormality is recorded as “Failure of light source for external light, contamination of diffusion index for external light” and the matters of which a user should be informed is recorded as “Please clean index”. That is, the determination unit 292 determines that an abnormality has occurred in the diffused light source unit 434 or diffusion index member 432 if the characteristic value of the measurement history for the measured values of the external light calculated by the calculation unit 291 is of the falling trend and the characteristic value of the measurement history for the measured values of the illumination light is normal. In this case, the display control unit 294 causes the clean lamp 26b of the display unit 26 to be lighted and causes the display monitor 26a to display information indicating the content of abnormality. For “5” of the abnormality information table T1, if no improvement is achieved even if cleaning of the index is performed, because maintenance of the light source unit 22 becomes necessary, as the matters of which the user should be informed, “Please contact serviceman” is recorded.

For “6” of the abnormality information table T1, if the characteristic value of the measurement history for the measured values of the external light is of a “rising trend” and the characteristic value of the measurement history for the measured values of the illumination light is of a “rising trend”, the predicted content of abnormality is recorded as “Presence of impurity of high reflectivity on both indices, abnormality in first light receiving unit” and the matters of which a user should be informed is recorded as “Please clean index”. That is, the determination unit 292 determines that any one of an abnormality in the reflection index member 422, an abnormality in the diffusion index member 432, and an abnormality in the first light receiving unit 24 has occurred, if the characteristic value of the measurement history for the measured values of the external light calculated by the calculation unit 291 is of the rising trend and the characteristic value of the measurement history for the measured values of the illumination light is of the rising trend. In this case, the display control unit 294 causes the call lamp 26c of the display unit 26 to be lighted and causes the display monitor 26a to display information indicating the content of abnormality. For “6” of the abnormality information table T1, if the characteristic value of each of the measured values of the external light and the measured values of the illumination light does not change even if cleaning of the indices is performed, “Please contact serviceman” is recorded as the matters of which a user should be informed.

For “7” of the abnormality information table T1, if the characteristic value of the measurement history for the measured values of the external light is of a “rising trend” and the characteristic value of the measurement history for the measured values of the illumination light is of a “falling trend”, the predicted content of abnormality is recorded as “Decrease in quantity of illumination light, abnormal output of external light source, abnormality in first light receiving unit, runaway of optical measurement apparatus” and the matters of which a user should be informed is recorded as “Please contact serviceman”. That is, the determination unit 292 determines that any one or more of an abnormality in the light source unit 22, an abnormality in the diffused light source unit 434, an abnormality in the first light receiving unit 24, and an abnormality in the optical measurement apparatus 2 has or have occurred, if the characteristic value of the measurement history for the measured values of the external light calculated by the calculation unit 291 is of the rising trend and the characteristic value of the measurement history for the measured values of the illumination light is of the falling trend. In this case, the display control unit 294 causes the clean lamp 26b of the display unit 26 to be lighted and causes the display monitor 26a to display information indicating the content of abnormality.

For “8” of the abnormality information table T1, if the characteristic value of the measurement history for the measured values of the external light is of a “falling trend” and the characteristic value of the measurement history for the measured values of the illumination light is of a “rising trend”, the predicted content of abnormality is recorded as “Failure of light source for external light, contamination of diffusion index for external light, adhesion of impurity of high reflectivity on reflection index for illumination light, runaway of optical measurement apparatus” and the matters of which a user should be informed is recorded as “Please call serviceman”. That is, the determination unit 292 determines that any one or more of failure of the diffused light source unit 434, an abnormality in the diffusion index member 432, an abnormality in the reflection index member 422, and an abnormality in the optical measurement apparatus 2 has or have occurred, if the characteristic value of the measurement history for the measured values of the external light calculated by the calculation unit 291 is of the falling trend and the characteristic value of the measurement history for the measured values of the illumination light is of the rising trend. In this case, the display control unit 294 causes the call lamp 26c of the display unit 26 to be lighted and causes the display monitor 26a to display information indicating the content of abnormality or failure.

For “9” of the abnormality information table T1, if the characteristic value of the measurement history for the measured values of the external light is of a “falling trend” and the characteristic value of the measurement history for the measured values of the illumination light is of the “falling trend”, the predicted content of abnormality is recorded as “Dust on both indices, failure of external light source or illumination light source” and the matters of which a user should be informed is recorded as “Please clean index”. The determination unit 292 determines that any one of failure of the diffused light source unit 434, an abnormality in the diffusion index member 432, an abnormality in the reflection index member 422, and an abnormality in the light source unit 22 has occurred, if the characteristic value of the measurement history for the measured values of the external light calculated by the calculation unit 291 is of the falling trend and the characteristic value of the measurement history for the measured values of the illumination light is of the falling trend. In this case, the display control unit 294 causes the clean lamp 26b of the display unit 26 to be lighted and causes the display monitor 26a to display information indicating the content of failure. For “9” of the abnormality information table T1, if the characteristic value of the measurement history of each of the measured values of the external light and the measured values of the illumination light does not change even if cleaning of the indices is performed, “Please contact serviceman” is recorded as the matters of which a user should be informed.

Accordingly, the determination unit 292 estimates, based on the abnormality information table T1 recorded by the abnormality information recording unit 272 and the characteristic values of the measurement histories calculated by the calculation unit 291, a location of any of degradation, abnormality, or failure of the optical measurement system 1.

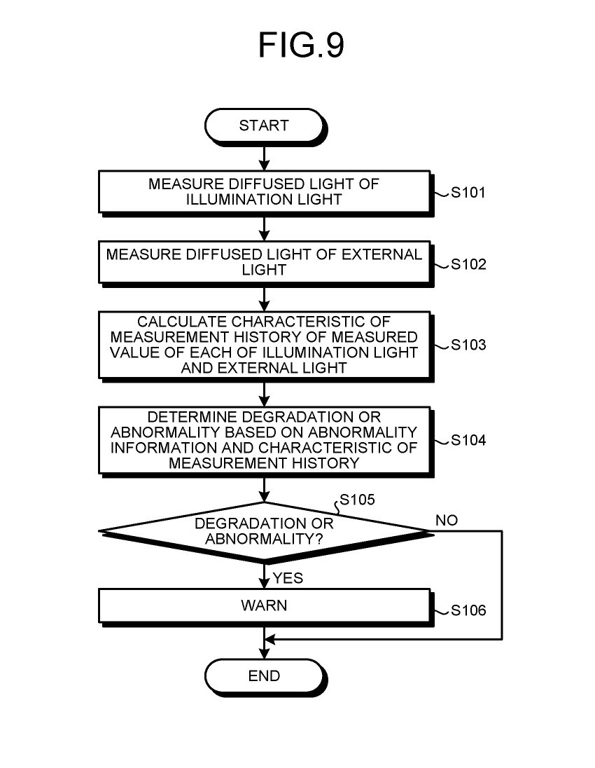

Next, a degradation and abnormality determination process executed by the optical measurement system 1 will be described. FIG. 9 is a flow chart illustrating an outline of the degradation and abnormality determination process executed by the optical measurement system 1.

As illustrated in FIG. 9, first, the control unit 29 causes the light source unit 22 to emit illumination light with the distal end portion 35 of the measurement probe 3 being inserted in the insertion portion 423a of the reflection index calibration unit 42, and causes the first light receiving unit 24 to receive and measure diffused light of the illumination light (step S101).

Subsequently, the control unit 29 causes the first light receiving unit 24 to receive and measure diffused light of external light with the distal end portion 35 of the measurement probe 3 being inserted in the insertion portion 433a of the emission index calibration unit 43 (step S102).

Thereafter, the calculation unit 291 calculates a characteristic value of a measurement history of measured values of each of the illumination light and the external light (step S103).

Subsequently, the determination unit 292 determines a degradation or an abnormality in the optical measurement system 1 based on abnormality information recorded in the abnormality information recording unit 272 and the characteristic values of the measurement histories calculated by the calculation unit 291 (step S104).

Thereafter, if the optical measurement system 1 is determined to have the degradation or the abnormality by the determination unit 292 (step S105: Yes), the display control unit 294 performs warning by causing the display unit 26 to display information indicating a content of the abnormality (step S106). Thereby, a user is able to know a location of the abnormality in the optical measurement system 1, for example, the degradation or the abnormality in the measurement probe 3 or the degradation or the abnormality in the light source unit 22. After step S106, the optical measurement system 1 ends this process.

If the optical measurement system 1 is determined not to have the degradation or the abnormality by the determination unit 292 in step S105 (step S105: No), the optical measurement system 1 ends this process.

According to the first embodiment of the present invention, because the determination unit 292 determines an abnormality in the optical measurement system 1 based on the characteristic value of the measurement history of each of the measured values of the external light and the measured values of the illumination light calculated by the calculation unit 291 and the abnormality information recorded by the abnormality information recording unit 272, occurrence of an abnormality in the measurement probe 3 or the light source unit 22 is able to be infallibly distinguished.

In the first embodiment of the present invention, although the display control unit 294 performs the warning on the display unit 26 based on the determination result of the determination unit 292, as long as the calculation result by the calculation unit 291 is able to be checked by a serviceman, only recording of the calculation result of the calculation unit 291 in the recording unit 27 may be performed. Thereby, the serviceman is able to accurately grasp the location of the abnormality in the optical measurement system 1 based on the calculation result of the calculation unit 291 recorded by the recording unit 27.

### Second Embodiment

Next, a second embodiment of the present invention will be described. An optical measurement system according to the second embodiment is different in configuration from the optical measurement system 1 according to the above described first embodiment. Specifically, a measurement probe has a plurality of light receiving fibers. Therefore, hereinafter, a configuration of the optical measurement system according to the second embodiment will be described. The same reference signs will be given to the same elements as those of the optical measurement system 1 of the above described first embodiment, and the explanation of the same elements will be omitted.

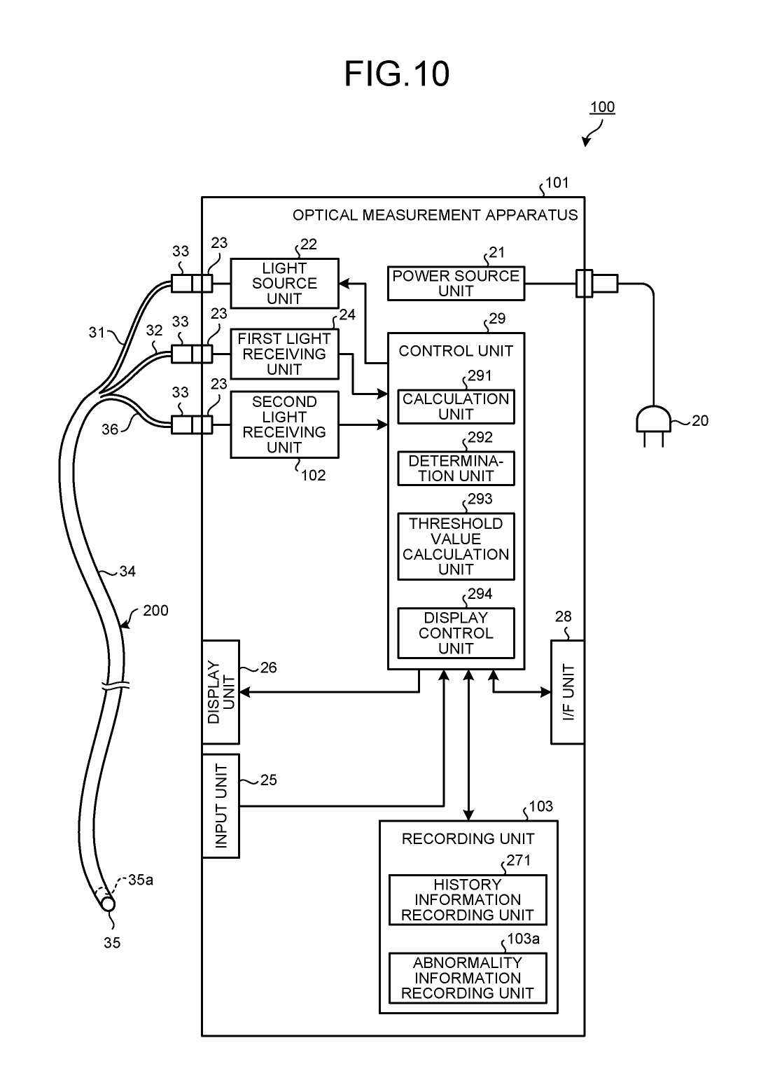

FIG. 10 is a block diagram illustrating a functional configuration of an optical measurement system 100 according to the second embodiment of the present invention.

The optical measurement system 100 illustrated in FIG. 10 includes: an optical measurement apparatus 101 that performs optical measurement on a measurement target, such as a body tissue that is a scatterer, and detects properties (characteristics) of the measurement target; and a measurement probe 200 that is freely attachable and detachable to and from the optical measurement apparatus 101 and is introduced into a subject.

First, a configuration of the optical measurement apparatus 101 will be described. The optical measurement apparatus 101 includes the commercial power source connector 20, the power source unit 21, the light source unit 22, the connection unit 23, the first light receiving unit 24, the input unit 25, the display unit 26, the I/F unit 28, the control unit 29, a second light receiving unit 102, and a recording unit 103.

The second light receiving unit 102 generates and outputs to the control unit 29, a result of measurement on the measurement target by receiving and performing photoelectric conversion on returned light of illumination light incident from the measurement probe 200 via the connection unit 23. The second light receiving unit 102 is realized by using an imaging element, such as a CCD or a CMOS. In the first light receiving unit 24, a spectrometer, which receives the returned light of the illumination light incident from the measurement probe 200 and is able to disperse spectral components of the received returned light of the illumination light, may be provided.

The recording unit 103 records therein various programs for operating the optical measurement apparatus 101 and various data or the like used for the optical measurement apparatus 101. Further, the recording unit 103 has the history information recording unit 271 that records a measurement result in a past calibration process, and has an abnormality information recording unit 103a that records abnormality information in which predicted abnormalities, characteristic values of measurement histories of each of the illumination light and external light estimated when these abnormalities occur, and contents displayed on the display unit 26 are associated with one another. The predicted abnormalities includes abnormalities due to optical performance of each of the measurement probe 200 and light source unit 22 and abnormalities due to structural defects of each of the measurement probe 200 and light source unit 22. The recording unit 103 is realized by using a volatile memory, a non-volatile memory, and the like. The recording unit 103 may be configured by using a memory card or the like inserted from outside of the optical measurement apparatus 101.

Next, a configuration of the measurement probe 200 will be described. The measurement probe 200 is configured by using at least three optical fibers. Specifically, the measurement probe 200 is configured by using the illumination fiber 31, the first light receiving fiber 32, and a second light receiving fiber 36. Further, the measurement probe 200 includes the proximal end portion 33, the flexible portion 34, and the distal end portion 35.

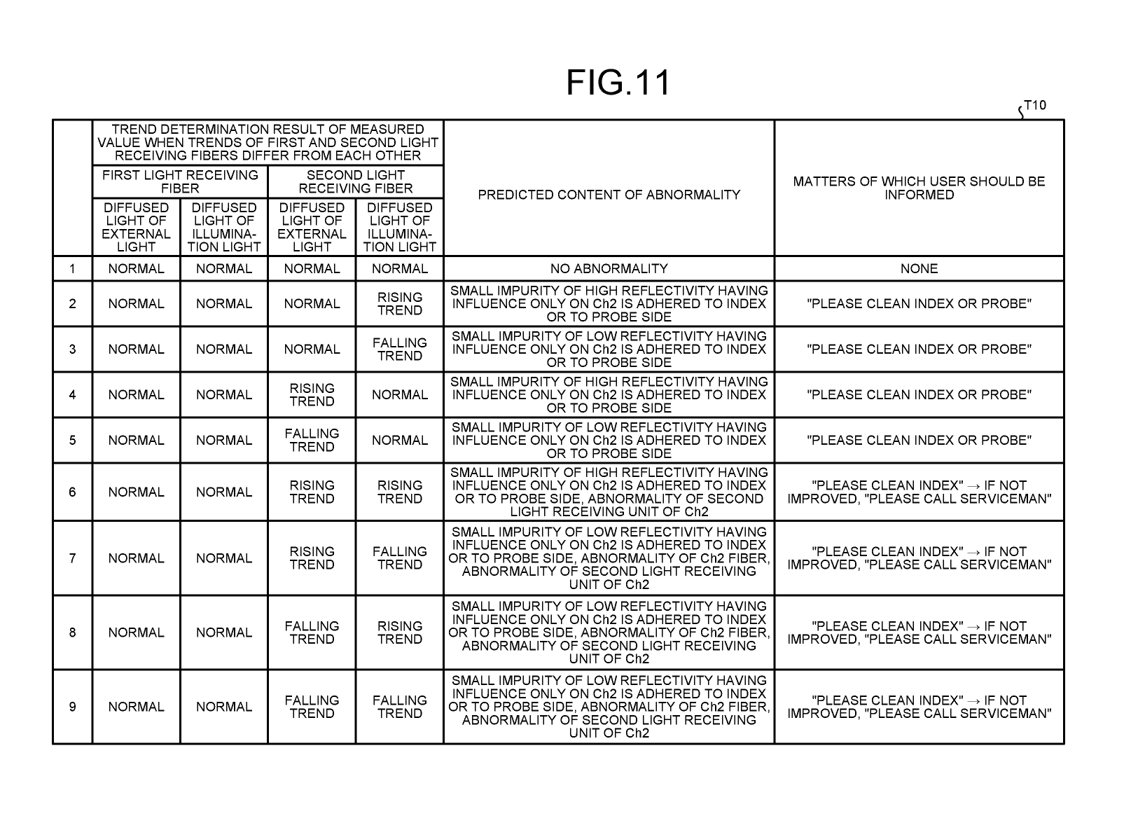

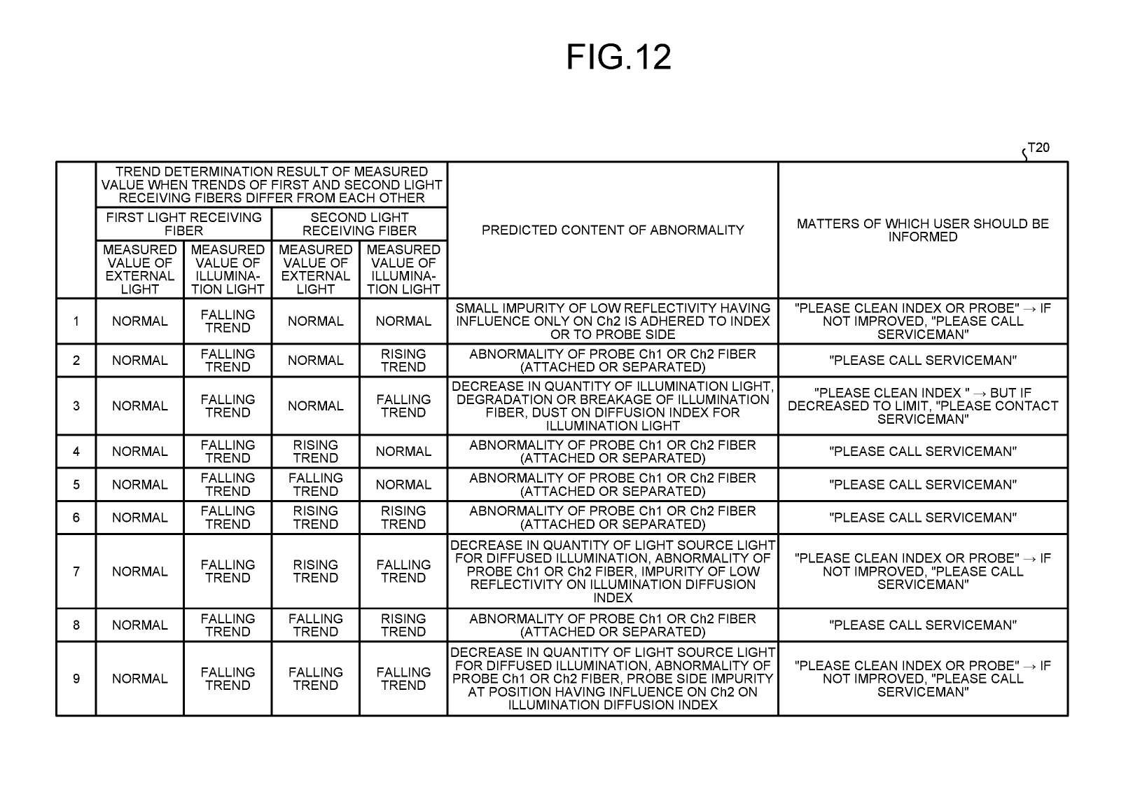

For the optical measurement system 100 configured as described above, degradation and abnormality patterns determined by the determination unit 292 will be described. FIG. 11 is a diagram illustrating an example of an abnormality information table related to abnormality information recorded by the abnormality information recording unit 103a. FIG. 12 is a diagram illustrating another example of the abnormality information table related to the abnormality information recorded by the abnormality information recording unit 103a.

In the abnormality information table T10 and the abnormality information table T20 illustrated in FIG. 11 and FIG. 12, the characteristic value of the measurement history of the measured values by the calculation unit 291 for each of the first light receiving fiber 32 and second light receiving fiber 36 (the characteristic value of the measurement history of each of the measured values of the external light and the measured values of the illumination light), predicted contents of abnormality, and matters of which a user should be informed are recorded in association with one another. Specifically, in the abnormality information table T10, for the nine patterns of the first light receiving fiber 32 described above in the first embodiment, the same number of measurement results of the second light receiving fiber 36 exist, and thus a total of 81 patterns exist. Therefore, hereinafter, the representative two examples illustrated in FIG. 11 and FIG. 12 will be described.

The abnormality information table T10 illustrated in FIG. 11 illustrates an example in which the characteristic values of the measurement histories of the measured values of the external light and measured values of the illumination light in the second light receiving fiber 36 respectively change when the characteristic value of the measurement history of each of the measured values of the external light and measured values of the illumination light in the first light receiving fiber 32 is normal.

Specifically, in the abnormality information table T10, the characteristic values of the measurement histories for each of the measured values of the first light receiving fiber 32 and second light receiving fiber 36 by the calculation unit 291, the predicted contents of abnormality, and the matters of which a user should be informed are recorded in association with one another. For example, as illustrated in the abnormality information table T10, if the characteristic values of the measurement histories of the measured values of the first light receiving fiber 32 and second light receiving fiber 36 differ from each other, presence of an impurity influencing only the second light receiving fiber 36 is possible. Therefore, for a light quantity of the diffused light source unit 434 and a light quantity of the light source unit 22, since a trend of the first light receiving fiber 32 is normal, the diffused light source unit 434 and light source unit 22 are considered to be normal, and the optical measurement apparatus 101 is also considered to be normal. That is, the determination unit 292 determines that an abnormality has occurred in the second light receiving fiber 36 or the second light receiving unit 102. In this case, the display control unit 294 causes the clean lamp 26b of the display unit 26 to be lighted and causes the display monitor 26a to display information indicating the content of abnormality. For “6” to “9” of the abnormality information table T10, if no improvement is achieved even if cleaning of the index is performed, “Please call serviceman” is recorded as the matters of which a user should be informed.

Further, the abnormality information table T20 illustrated in FIG. 12 illustrates an example in which the characteristic value of the measurement history of the measured values of the illumination light in the first light receiving fiber 32 indicates a falling trend.

Specifically, in the abnormality information table T20, the characteristic values of the measurement histories for each of the measured values of the first light receiving fiber 32 and second light receiving fiber 36 by the calculation unit 291, the predicted contents of abnormality, and the matters to be informed to a user are recorded in association with one another. For example, as illustrated for “3” of the abnormality information table T20, if the measured values of the external light are normal and the measured values of the illumination light have a falling trend, in both the first and second light receiving fibers, the determination unit determines that an abnormality has occurred, such as a decrease in illumination light quantity, degradation or breakage of the illumination fiber, or dust on the diffusion index for the illumination light. In this case, the display control unit 294 causes the clean lamp 26b of the display unit 26 to be lighted and causes the display monitor 26a to display information indicating the content of abnormality. The determination unit 292 determines that an abnormality has occurred in the second light receiving fiber 36 if the characteristic value of the measured values of the second light receiving fiber 36 is of a falling trend or rising trend. In this case, the display control unit 294 causes the call lamp 26c of the display unit 26 to be lighted and causes the display monitor 26a to display information indicating the content of abnormality.

As described above, in the abnormality information recording unit 103a, 81 kinds of the above described pattern are recorded. The determination unit 292 determines degradation or an abnormality in the optical measurement system 100 based on the characteristic value of the measurement history of the measured values of each of the first light receiving fiber 32 and second light receiving fiber 36 calculated by the calculation unit 291 and the abnormality information recorded by the abnormality information recording unit 103a. Thereby, degradation or an abnormality is able to be determined per light receiving fiber.

According to the above described second embodiment of the present invention, the determination unit 292 is able to determine an abnormality in the first light receiving fiber 32 and second light receiving fiber 36 individually, based on the characteristic value of the measurement history of each of the measured values of the external light and the measured values of the illumination light calculated by the calculation unit 291 and the abnormality information recorded by the abnormality information recording unit 103a.

In the above described second embodiment, although the measurement probe 200 includes the first light receiving fiber 32 and second light receiving fiber 36, the number of light receiving fibers may be changed as appropriate and for example, three or more may be included. Of course, the number of the illumination fibers 31 may be changed as appropriate.

According to an optical measurement apparatus of some embodiments, it is possible to accurately distinguish between an abnormality due to an optical performance and an abnormality due to a structural defect.

Accordingly, the present invention may include various embodiments not described herein, and various design changes or the like within the scope of the technical ideas specified by the scope of the claims may be made.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An optical measurement apparatus to which a measurement probe having an illumination fiber and a light receiving fiber is configured to be detachably connected and which is configured to perform optical measurement on a measurement target, the optical measurement apparatus comprising:
a light source unit configured to emit illumination light to the measurement target via the illumination fiber;
a light receiving unit configured to receive light propagated via the light receiving fiber; and
a calculation unit configured to calculate a characteristic value of a measurement history of each of the illumination light and external light incident from outside, based on a plurality of measured values obtained by receiving, by use of the light receiving unit via the light receiving fiber, diffused light of the illumination light reflected from a reflection index member and on a plurality of measured values obtained by receiving, by use of the light receiving unit via the light receiving fiber, diffused light of the external light that has been transmitted through a diffusion index member as uniform light.

2. The optical measurement apparatus according to claim 1, further comprising:
an abnormality information recording unit configured to record abnormality information in which predicted abnormalities due to an optical performance and due to a structural defect in each of the measurement probe and the light source unit are associated with the characteristic value of the measurement history of each of the illumination light and the external light estimated when the abnormalities occur; and
a determination unit configured to determine whether or not an abnormality has occurred in the measurement probe or the light source unit, based on the characteristic value of the measurement history calculated by the calculation unit and on the abnormality information.

3. The optical measurement apparatus according to claim 2, further comprising an output unit configured to output a warning when the determination unit determines that the abnormality has occurred in the measurement probe or the light source unit.

4. The optical measurement apparatus according to claim 3, wherein
the measurement probe further comprises one or more additional light receiving fibers, and
the determination unit is configured to determine whether or not the abnormality has occurred for each of the light receiving fiber and the one or more additional light receiving fibers.
